(19) United States
(12) Patent Application Publication
(10) Pub. No.: US 2017/0159017 A1
(43) Pub. Date: Jun. 8, 2017

(54) Method For Introducing Exogenous Mitochondria Into A Mammalian Cell

(71) Applicant: GUANGZHOU INSTITUTES OF BIOMEDICINE AND HEALTH, CHINESE ACADEMY OF SCIENCES, Guangzhou, Guangdong (CN)

(72) Inventors: Xingguo Liu, Guangzhou (CN); Duanqing Pei, Guangzhou (CN); Jinglei Liu, Guangzhou (CN); Deyang Yao, Guangzhou (CN); Xuebin Liu, Guangzhou (CN)

(73) Assignee: Guangzhou Institutes of Biomedicine and Health, Chinese Academy of Sciences, Guangzhou (CN)

(21) Appl. No.: 15/034,687

(22) PCT Filed: Aug. 25, 2014
(86) PCT No.: PCT/CN2014/085117

§ 371 Date: May 5, 2016

(30) Foreign Application Priority Data
Nov. 8, 2013 (CN) 201310554218.7

(51) Int. Cl. C12N 5/0786; C12N 15/87

(52) U.S. Cl. CPC C12N 5/0645; C12N 15/87; C12N 2510/00

(57) ABSTRACT

The present disclosure provides a method for producing a cell with exogenous mitochondria by obtaining synthetic mitochondria via introduction of exogenous mitochondrial DNA into mitochondria or empty mitochondrial shells, and incorporating the same into mammalian cells via endocytosis. As such, effective functionality of exogenous mitochondria in cells is realized. The synthetic mitochondrial DNA genes introduced according to the present disclosure can be stably expressed and effectively passaged. The method for introducing exogenous mitochondrial DNA into mammalian cells as disclosed herein may be used as a whole new mitochondrial molecular cloning means to perform site-directed mutagenesis, gene insertion, gene knockout, gene rearrangement, and the like in mitochondria. Therefore, any molecular cloning modification can be performed on a mammalian mitochondrial DNA, which is of great importance to therapeutic schemes of diseases derived from mitochondrial DNA mutations.

### TECHNICAL FIELD

The present disclosure relates to the field of biological and genetic engineering, especially a method for introducing exogenous mitochondria into a mammalian cell.

### BACKGROUND

Mitochondria are the most important organelles in eukaryotes, responsible for more than 90% of cellular energy supply. Mitochondria carry an independent genome, i.e., mitochondrial DNA, with independent gene transcription and protein translation configurations that are different from the nuclear genome. Mammalian mitochondrial DNA encodes 22 tRNAs, 2 rRNAs, and 13 polypeptides. These encoded polypeptides are critical subunits in a variety of protein complexes involved in aerobic respiration of mitochondria. A large number of studies have shown that mutations or reduced expression of these polypeptides can significantly inhibit cell aerobic respiration.

With the rapid development of modern molecular biology, people have been able to conduct a variety of genetic modification activities in the nuclear genome, such as gene knockin, gene knockout, site-directed mutagenesis, gene rearrangement, and so on. However, as of now, mitochondrial genetic modification technology is still very immature. People have only achieved mitochondrial genetic modification in yeast and other lower eukaryotes, but not yet in higher mammals. Many efforts have been reported regarding mitochondrial modification in mammals, but overall, none has been actually successful. One attempt was to express target genes in the nuclear genome, add localization sequences, and then introduce into mitochondria. There are problems with this approach. First of all, the majority of mammalian mitochondrial genome encodes functional RNAs, while the mechanism for RNAs encoded by nuclear genome to enter mitochondria has not been well understood. In a known mechanism, it is required to add a localization RNA sequence, yet it is unknown whether such a sequence would affect the functionality of target RNAs. On the other hand, after millions of years of evolution, the 13 polypeptides that currently remain in the mammalian mitochondrial genome differ from most nuclear genes. Expression of such polypeptides in nuclear genome may induce a problem of cytotoxicity. In addition, these polypeptides are often highly hydrophobic, which is the disadvantage of nuclear genome expression. Another attempt was to develop specific transfection media for mitochondria so as to transfect the target genes into mitochondria. Much has been reported in the literature in this aspect, but it is very difficult to transfer a target nucleic acid into mitochondria across its three-layer membrane structure from outside the cell. Few success is reported for methods using, for example, dequalinium plastids (DQAsome), polyethyleneimine (PEI) and derivatives. The methods reported also have few citations with poor reproducibility, and therefore cannot really achieve their goals. A third attempt was the use of techniques of nuclear transfer or cytoplasmic body hybridization, to increase the proportions of exogenous wild-type mitochondria in order to engineer the genetic behavior of mitochondria. Such methods cannot eradicate endogenous mitochondria and DNA, while it may additionally increase large amounts of exogenous cytoplasmic components and thus further increase uncertainty. Yet another attempt was to express TALEN molecules targeting mitochondria to disrupt the target sequence. This approach has been reported to successfully destroy mitochondrial DNA which has large deletions and specific point mutations. Being the most effective technical process for modifying mitochondrial DNA reported, this method is still insufficient in many ways. On one hand, for specific point mutations, it is highly doubted whether TALEN can specifically select a target sequence. On the other hand, this method can only achieve focused destruction of specific sequences, yet not site directed modifications or gene knockin. Mitochondria was discovered a hundred years ago, while mitochondrial DNA has been known for several decades. Efforts have been made aimed at mitochondrial DNA transformation ever since. However, until now, scientists have not been able to perform effective genetic modification of mammalian mitochondrial DNA. Effective mitochondrial genetic modification technique in mammalian cells thus remains a world-class technical problem.

The Craig J. Venter lab succeeded in obtaining synthetic whole sequences of mouse mitochondrial DNA with techniques in synthetic biology, but has not made any modifications thereto. Nor were they able to express the synthetic mitochondrial DNA in mammalian cells or identify the functionalities thereof.

### SUMMARY OF THE INVENTION

Therefore, according to one aspect of the present disclosure, a mammalian cell comprising exogenous mitochondria is provided.

Specifically, the following technical solutions are disclosed.

A mammalian cell comprising exogenous mitochondrion is disclosed, wherein the cell has the function of endocytosis.

The exogenous mitochondrion herein may be a synthetic mitochondrion obtained by introducing an exogenous DNA into a mitochondrion or an empty mitochondrial shell, or it may be an isolated mammalian cellular mitochondrion.

The exogenous DNA may be a synthetic mitochondrial DNA, or it may be an isolated DNA. In one of the embodiments, the exogenous DNA is a synthetic mitochondrial DNA from any source.

In one of the embodiments, the synthetic mitochondrial DNA may be a mammalian cellular mitochondrial DNA with artificial gene modification. Of course, the synthetic mitochondrial DNA may be a wild-type mammalian cellular mitochondrial DNA as well.

The term “mammalian cell” used herein refers to a cell with endocytosis function, most preferably a macrophage.

According to another aspect of the present disclosure, a method for preparing a mammalian cell comprising exogenous mitochondria aforementioned is provided.

Specifically, the following technical solutions are disclosed.

The method for preparing a mammalian cell comprising exogenous mitochondrion may comprise adding the exogenous mitochondrion to a cell culture system comprising a mammalian cell with endocytosis function and continuing culturing to obtain cells with the exogenous mitochondrion.

In one of the embodiments, the mammalian cell may be a macrophage. The culturing conditions may be 36-38° C. in temperature and 4.5-5.5% CO₂ in a water-saturated closed incubator.

According to yet another aspect of the present disclosure, a synthetic mitochondrion is provided.

Specifically, the following technical solutions are disclosed.

A synthetic mitochondrion is provided, which may be obtained by introducing an exogenous DNA into a mitochondrion or an empty mitochondrial shell.

Method for preparing the synthetic mitochondrion aforementioned is also provided.

Accordingly, the method for preparing the synthetic mitochondrion may comprise:

obtaining an exogenous DNA;

preparing an empty mitochondrial shell, or obtaining a mitochondrion by isolation; and

introducing the exogenous DNA obtained from Step (1) into the empty mitochondrial shell or into the isolated mitochondrion obtained from Step (2) via eletroporation to obtain the synthetic mitochondrion.

In one of the embodiments, the exogenous DNA may be an isolated wild-type DNA. Or, the exogenous DNA may be a synthetic mitochondrial DNA, which is prepared by the following steps:

designing a new DNA sequence composition based on a mammalian mitochondrial DNA sequence using at least one gene modification means selected from the group consisting of gene introduction, gene knockout, site-directed mutagenesis, and gene rearrangement as needed, or, obtaining a wild-type DNA sequence composition; and

synthesizing DNA fragments of 50 bp-60 bp according to the DNA sequence composition and connecting the DNA fragments synthesized with Gibson isothermal one-step method to obtain the synthetic mitochondrial DNA.

According to yet another aspect of the present disclosure, a method for introducing exogenous mitochondria into a mammalian cell is provided.

Specifically, the following technical solutions are disclosed.

Accordingly, the method for introducing exogenous mitochondria into a mammalian cell may comprise:

(1) obtaining an exogenous mitochondrion; and

(2) adding the exogenous mitochondrion obtained from Step (1) to a cell culture system comprising mammalian cells with endocytosis function and continuing culturing to obtain cells with the exogenous mitochondrion.

In one of the embodiments, the exogenous mitochondrion may be an isolated mammalian cellular mitochondrion.

In one of the embodiments, the exogenous mitochondrion may be a synthetic mitochondrion obtained by introducing an exogenous DNA into a mitochondrion or an empty mitochondrial shell.

In one of the embodiments, the exogenous DNA may be an isolated wild-type DNA.

In one of the embodiments, the exogenous DNA may be a synthetic mitochondrial DNA, which may be prepared by the following steps:

(1) designing a new DNA sequence composition based on a mammalian mitochondrial DNA sequence using at least one gene modification means selected from the group consisting of gene introduction, gene knockout, site-directed mutagenesis, and gene rearrangement as needed, or, obtaining a wild-type DNA sequence composition; and

(2) synthesizing DNA fragments of 50 bp˜60 bp according to the DNA sequence composition and connecting the DNA fragments synthesized with Gibson isothermal one-step method to obtain the synthetic mitochondrial DNA.

In one of the embodiments, the mammalian cell may be a macrophage.

In one of the embodiments, the culturing conditions may be 36-38° C. in temperature and 4.5-5.5% CO₂ in a water-saturated closed incubator.

The present disclosure utilizes synthetic biology to artificially synthesize a whole mitochondrial DNA sequence, realizing a fusion expression of GFP and COX—I based on a wild-type sequence, which provides a simple and effective means for molecular cloning of mitochondrial genome. We have successfully developed, for the first time, a method for introducing an exogenous mitochondrial DNA into a cell via endocytosis by a macrophage and obtained a synthetic mitochondrial DNA cell with stable GFP expression and stable passage.

The present disclosure is the first time in the world that molecular cloning operation has been successfully performed in mammalian mitochondrial DNA. The present disclosure introduces exogenous mitochondrial DNA into cells via endocytosis by macrophage, resulting in stable expression of genes from the exogenous mitochondrial DNA in mammalian cells. Such cells can be effectively passaged. Therefore, effective functionality of exogenous mitochondria in cells can be realized. The method for introducing exogenous mitochondrial DNA into mammalian cells as disclosed herein may be used as a whole new mitochondrial molecular cloning means to perform site-directed mutagenesis, gene insertion, gene knockout, gene rearrangement, and the like in mitochondria. Therefore, any molecular cloning modification can be performed on a mammalian mitochondrial DNA with high purity and without any limitation in sources, which is of great importance to therapeutic schemes of diseases derived from mitochondrial DNA mutations.

### BRIEF DESCRIPTION OF THE DRAWINGS

FIG. 1 shows the result of exogenous mitochondria entering a macrophage as described in Example 1. 1A shows a DsRed2 detection result thereof, showing exogenous mitochondria originated from NIH3T3. 1B shows a corresponding EYFP detection result, showing endogenous mitochondria within the macrophage. 1c shows a result of two-color overlay.

FIG. 2 shows the in vitro assembly of synthetic mitochondria as described in Example 3. 2A shows the detection of properly transcript within the assembled synthetic mitochondria. 2B shows the detection of DNA replication within the assembled synthetic mitochondria. Description of symbols: M: molecular weight marker; Positive: positive control; Assembly: assembled synthetic mitochondria; Negative: negative control.

FIG. 3 shows the fusion expression of GFP-COX I in mouse macrophage RAW264.7 as described in Example 4. 3A shows the locations of all mitochondria in the cell stained by Mito-Tracker Red dye. Mito-Tracker Red is a mitochondria-specific dye. 3B shows the GFP signal of the green GFP-COX I fusion gene. 3C shows the DAPI staining results, indicating the location of the nucleus. 3D is a result of three-color overlay.

### DETAILED DESCRIPTION OF THE INVENTION

The term “genetic modification” as used herein refers to construction of desoxyribonucleic acid (DNA) molecules of a target sequence via in vitro designing and total synthesis techniques. Genetic modifications as used herein involves designing a new DNA sequence, which may be exactly the same as a wild-type sequence or with any sequence modification as needed, including site-directed mutagenesis, gene introduction, gene knockout, and gene rearrangement.

Endocytosis, also known as endocytosis effect, is a process of transporting extracellular materials into a cell by the deformation movement of plasma membrane. Depending on the various size of engulfed substances and different endocytosis mechanisms, endocytosis can be divided into three types: phagocytosis, pinocytosis, and receptor-mediated endocytosis.

In order to facilitate clear understanding of the technical contents of the present disclosure, the following specific embodiments are described in details below with reference to the accompanying drawings. It should be understood that these embodiments are intended only for the purpose of illustrating the present invention, without any limitation to the scope thereof. Specific experimental methods that are not explicitly noted in the following examples are generally in accordance with conventional conditions, for example, as described in Sambrook et al., Molecular Cloning: A Laboratory Manual (New York: Cold Spring Harbor Laboratory Press, 1989), or according to the manufacturer's recommendations. All the chemical reagents involved in the embodiments are commercially available products.

### Example 1

This embodiment demonstrates that exogenous mitochondria may enter into mammalian cells by endocytosis. The endogenous mitochondria of the macrophages were labeled with EYFP by stably expressing fluorescent protein EYFP localized in mitochondria in macrophages. The endogenous mitochondria of the NIH3T3 cells were labeled with DsRed2 by stably expressing fluorescent protein DsRed2 localized in mitochondria in NIH3T3 cells. The NIH3T3 mitochondria labeled with DsRed2 were isolated and added to a macrophage culture system. Observations were made using a confocal microscope after 12 hours. It was observed that mitochondria labeled with DsRed2 entered macrophages and presented the same morphology as the endogenous mitochondria of the macrophages labeled with EYFP. Specifically:

1. Mouse macrophage cell line RAW264.7 cells and NIH3T3 cell culture medium (high glucose DMEM (commercially available from Hyclone, Item No: SH30022.01B); 10% fetal bovine serum (commercially available from GIBCO, Item No: 16000-044); double antibody (including 10000 U/ml penicillin and 10000 μg/ml streptomycin, 1:100 dilution for use, available from GIBCO, Item No: 15140-122)). Cells were cultured in culture dishes according to conventional methods.

2. Plasmids mitoDsRed2 and mitoEYFP were constructed by means of molecular cloning. A mitochondrial localization sequence signal (MSVLTPLLLRGLTGSARRLPVPRAKIHSL) was attached to the N-terminus with standard DsRed2 (GenBank: AFS63392.1) and EYFP (GenBank: ACO48266.1) fluorescent protein sequences. The plasmids were constructed using pMXs vectors. Corresponding cells were transfected with the plasmids by electroporation and cultured via flow sorting monoclone culture to obtain cell lines stably expressing the fluorescent proteins.

3. The mitochondria were isolated with the use of Mitochondria/Cytosol Isolation Kit (APPLYGEN):

(1) the cells were washed once with PBS, centrifuged at 6000 rpm for 5 min, and had the supernatant discarded;

(2) cell pellet was collected from the tube bottom, re-suspended as cell suspension with 1.5 ml Mito-Cyto buffer pre-cooled with ice, and mixed well, for example, via suction and discharge by 5 ml syringe with extra small needle for about 40 times to yield cell homogenates;

(3) the cell homogenate from above was transferred to a centrifuge tube and centrifuged at 800×g under 4° C. for 5 min, allowing the nucleus, large membrane fragments, unlysed cells, and the like to deposit to the bottom of the tube;

(4) the pellet was discarded and the supernatant transferred to a new centrifuge tube, centrifuged again at 800×g under 4° C. for 5 min; and

(5) the pellet was discarded and the supernatant transferred to a new centrifuge tube, centrifuged to 12,000×g under 4° C. for 10 min, with the empty mitochondria shells precipitated in the bottom of the tube.

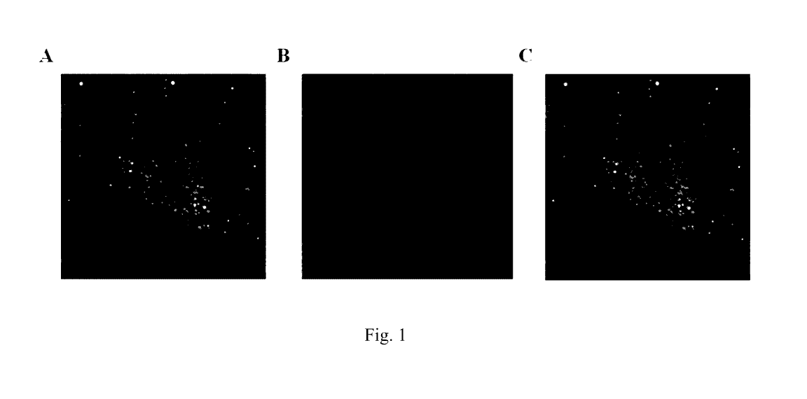

4. The resultant precipitated mitochondrial shells from above were re-suspended with 20 μL RAW264.7 cell culture medium. Aliquots of the suspension were added to culture dishes and incubated under 37° C. and 5% CO₂ in a water-saturated closed incubator. Observations could be made in 12 hours after endocytosis, and the results are shown in FIG. 1.

### Example 2

In this embodiment, a circular mitochondrial DNA of designed sequence, i.e., a circular mitochondrial DNA containing GFP-COX-I fusion gene, is obtained by gene introduction, i.e., insertion, of GFP and Linker sequences, primer synthesis, and DNA splicing, to mouse mitochondrial DNA.

Designing a New Artificial Circular Mitochondrial DNA:

In this embodiment, a circular mitochondrial DNA is obtained by gene transfer, i.e., insertion, of GFP and Linker sequences, primer synthesis, and DNA splicing, to mouse mitochondrial DNA. Specifically, the sequence was derived from a known mitochondrial DNA sequences of wild type C57 BL/6J mice (source of sequence: NCBI GenBank: EF108336) and GFP gene and Linker sequences were inserted in the position 5328 (specifically, as shown in Seq ID No.1), to obtain a designed circular mitochondrial DNA around 5 Kb containing the GFP-COX-I fusion gene.

2. Depending on the specific composition of the designed circular mitochondrial DNA from above, conventional primer synthesis methods were used to obtain a plurality of DNA fragments to be spliced, which were around 50 bp˜60 bp.

3. DNA splicing:

(1) Based on the prior art, the Gibson isothermal one-step method was used for DNA splicing. The enzymatic systems employed in the Gibson isothermal one-step method were prepared as follows: mixing 320 μl 5×ISO buffer (25% PEG-8000, 500 mM Tris-HCl pH 7.5.50 mM MgCl2, 50 mM DTT, 4 dNTPs of 1 mM each, 5 mM NAD), 0.64 μl 10 U/μl T5 exonuclease Epicentre, 20 μl 2 U/μl Phusion polymerase, and 160 μl 40 U/μl Taq ligase, filling with sterile water up to 1.2 ml, packing, and storing under −20° C. before use;

(2) In the Gibson isothermal one-step method, the fragments to be spliced were mixed in equal proportions with a final concentration of 2 ng/μl. 5 μl of the mixture were mixed evenly with 5 μl of the enzyme system described above and incubated at 50° C. for 1 hour to get properly spliced fragments.

4. The DNA was collected and purified. The spliced fragments were confirmed by sequencing to be the designed circular mitochondrial DNA above.

### Example 3

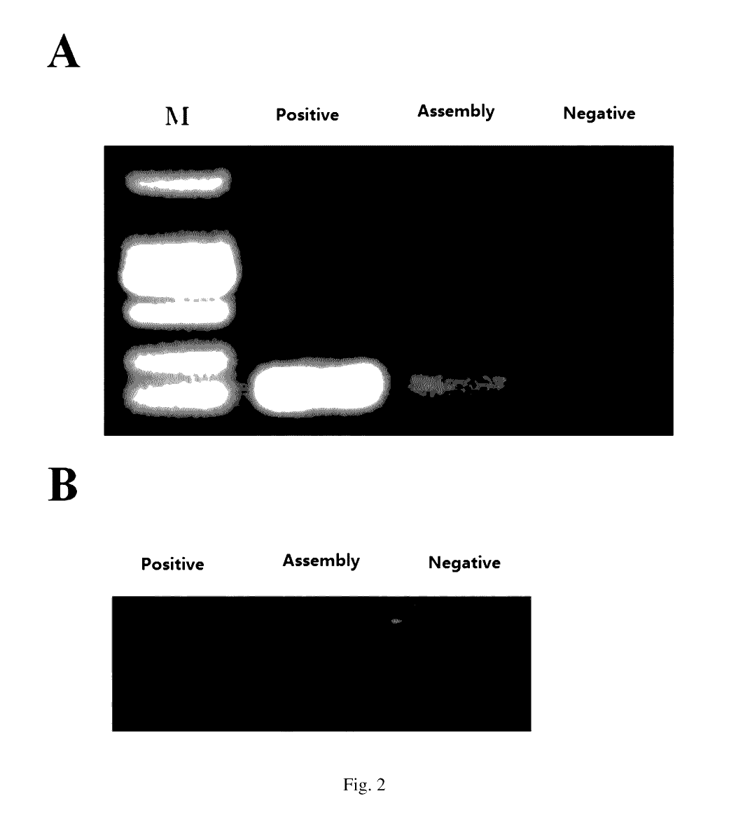

In this embodiment, the process of producing synthetic mitochondria through in vitro assembly of the circular mitochondrial DNA obtained in Example 2 and empty mitochondrial shells of NIH3T3 Rho0 cells is described, as well as the extraction and identification processes of the RNA and DNA of the synthesized mitochondria. FIG. 2 shows the detection of proper transcript (FIG. 2A) and DNA replicates (FIG. 2B) in the synthetic mitochondria.

I. Culturing of the Mitochondrion DNA-Free Rho0 Free Cells:

1. 1.5 μg/mL ditercalinium or 250 ng/mL ethidium bromide, 50 μg/mL uridine (Sigma), and 110 μg/mL sodium pyruvate were added to NIH3T3 cell culture medium;

2. Incubation was performed continuously for a month according to conventional methods;

3. NIH3T3 Rho0 medium (high glucose DMEM, 10% fetal bovine serum, 50 μg/mL uridine, 110 μg/mL sodium pyruvate, double antibody of penicillin and streptomycin) was used for culturing;

4. Cells were collected, i.e., NIH3T3 Rho0 cells without mitochondrial DNA were obtained.

II. Isolation of Empty Rho0 Mitochondrial Shells:

The mitochondria were isolated with the use of Mitochondria/Cytosol Isolation Kit (APPLYGEN):

(1) the cells were washed once with PBS, centrifuged at 6000 rpm for 5 min, and had the supernatant discarded;

(2) cell pellet was collected from the tube bottom, re-suspended as cell suspension with 1.5 ml Mito-Cyto buffer pre-cooled with ice, and mixed well, for example, via suction and discharge by 5 ml syringe with extra small needle for about 40 times to yield cell homogenates;

(3) the cell homogenate from above was transferred to a centrifuge tube and centrifuged at 800×g under 4° C. for 5 min, allowing the nucleus, large membrane fragments, unlysed cells, and the like to deposit to the bottom of the tube;

(4) the pellet was discarded and the supernatant transferred to a new centrifuge tube, centrifuged again at 800×g under 4° C. for 5 min; and

(5) the pellet was discarded and the supernatant transferred to a new centrifuge tube, centrifuged to 12,000×g under 4° C. for 10 min, with the empty mitochondria shells precipitated in the bottom of the tube.

III. Assembly of the Mitochondrial Shells and DNA Sequences (Electroporation):

(1) The empty mitochondrial shells were re-suspended with 50 μl electroporation buffer (0.33M sucrose, 10% glycerol);

(2) 10 μg mitochondrial genome (10 μl TE buffer for the blank group) was added to the tube and mixed well;

(3) The well-mixed suspension was transferred into 1 mm cuvette for an electric shock (parameters: field strength: 12˜16 Kv/cm; capacitance: 25 μF; and, resistance: 400Ω);

(4) After the shock, 1 ml Incubation buffer 1 (40 mM Tri-HCl 7.4; 25 mM NaCl; 5 mM MgCl2; 10% glycerol) was immediately added to the cuvette and mixed well with a pipette;

(5) The mixture was transferred to a new 2 ml centrifuge tube and centrifuged at 21000 g/min under 4° C. for 10 min, and the supernatant was discarded;

(6) The precipitation was washed twice using 1 ml Incubation buffer, respectively.

IV. RNA Extraction, Reverse Transcription, and Identification of the Synthetic Mitochondria:

1. Initial Extraction of the Synthetic Mitochondrial RNA:

(1) The pellet was re-suspended with 50 μl Incubation buffer 2 (40 mM Tri-HCl 7.4; 25 mM NaCl; 5 mM MgCl2; 10% glycerol; 1 mM pyruvate; 1 mM ATP; 1 mg/ml BSA) and incubated at 37° C. for 3 h;

(2) The culture from the above step (1) was added 1 ml Incubation buffer 2, washed three times, and centrifuged at 1000 g/min under 4° C. for 10 min;

(3) The supernatant was removed to the greatest extent possible with a pipette, then the remaining material was re-suspended with 10 μl Roche DNase I, 5 μl 10×DNase I buffer (110 mM Tris-HCl 7.4; 32.5 mM MgCl2) and 35 μl Incubation buffer 2 and incubated under in 37° C. for 30 min;

(4) The culture from the above step (3) was washed three times with 1 ml washing buffer (10% glycerol; 10 mM Tri-HCl 7.4; 150 mM NaCl; 1 mM EDTA) and centrifuged at 1000 g/min under 4° C. for 10 min.

2. RNA Extraction of the Synthetic Mitochondria Using a Qiagen RNA Extraction Kit:

(1) The material from the centrifugation was re-suspended with 700 μl QIAzol Lysis Reagent, mixed evenly using a pipette, and stood at room temperature for 5 min;

(2) The suspension was combined with 140 μl chloroform, well shaken for 15 s, and stood at room temperature for 2-3 min;

(3) The mixture was centrifuged at 12000 g under 4° C. for 15 min;

(4) The supernatant was transferred to a new EP centrifuge tube (RNase free), combined with 1.5× absolute ethanol, and mixed by pipetting;

(5) The mixed solution was added to the spin column (column volume: 700 μl; the remnant can be added in several times), and centrifuged at greater than 8000 g at room temperature for 15 s;

(6) 700 μl Buffer RWT was added before centrifuging at greater than 8000 g at room temperature for 15 s;

(7) 500 μl Buffer RPE was added before centrifuging at greater than 8000 g at room temperature for 15 s;

(8) 500 μl Buffer RPE was added before centrifuging at greater than 8000 g at room temperature for 2 min;

(9) A new 2 ml collection tube was used before centrifuging at 21000 g/min at room temperature for 1 min;

(10) The spin column was transferred to a new 1.5 mlEP tube, combined with 40 μl of RNase-free water, and centrifuged at greater than 8000 g at room temperature for 1 min;

(11) The eluted RNA in the EP tube was re-applied to the column and centrifuged at greater than 8000 g at room temperature for 1 min.

3. In Vitro Reverse Transcription of the Synthetic Mitochondrial RNA:

(1) Digestion of DNA in the RNA: conducted for 30 min with Promega DNase Iunder 37° C., with digestion system as follows:

| | |
|---|---|
| 10X DNase I buffer | 1 μl |
| DNase I | 1 μl |
| RNA | 8 μl |

(2) 1 μl stop buffer was added after digestion and the treatment lasted for 10 min under 65° C.;

(3) Reverse transcription of RNA: Digested RNA was treated for 5 min under 65° C., with the treatment system as follows:

| | |
|---|---|
| Digested RNA | 8 μl |
| Random primer | 1 μl |
| 10 mM dNTP | 1 μl |

Treated RNA was cooled on ice immediately after the treatment.

(4) The following reagents were added and mixed well:

| | |
|---|---|
| 10X RT buffer | 2 μl |
| 25 mM MgCl2 | 4 μl |
| 0.1 M DTT | 2 μl |
| RNase OUT | 1 μl |
| Super Script | 1 μl |

The following temperature treatments were done sequentially:

| | |
|---|---|
| 25° C. | 10 min |
| 50° C. | 50 min |
| 85° C. | 5 min |

Treated RNA was cooled on ice immediately after the treatment.

4. Identification of the Transcript in the cDNA Using PrimeSTAR Enzyme:

(1) The PCR reaction system is as follows:

| | |
|---|---|
| 5X PrimeSTAR ® Buffer (Mg²⁺ plus) | 10 μl |
| dNTP Mixture ( [symbol] 2.5 mM) | 4 μl |
| Primer 1 (10 μM) | 1 μl |
| Primer 2 (10 μM) | 1 μl |
| Template DNA | 2 μl |
| PrimeSTAR enzyme (2.5 U/μl) | 0.5 μl |
| Sterilized distilled water | 31.5 μl |

Reaction Conditions:

| | |
|---|---|
| 98° C. | 10 sec |
| 55° C. | 12 sec |
| 72° C. | 1 min |

Number of cycles: 30.

(2) The PCR product was subjected to 1.5% agarose gel electrophoresis, and photographs were taken for detection. The obtained results shown in FIG. 2A, wherein the “positive” lane represents the PCR product obtained using, as a template, the cDNA that was the reverse-transcript of the isolated RNA from the NIH3T3 cells. The “assembly” lane represents the PCR product obtained using, as a template, the cDNA that was electrically isolated after electroporation. The “negative” lane represents the lane with the PCR product obtained using, only water as a template.

V. In Vitro Replication and Detection of the Synthetic Mitochondrial DNA:

1. The turned mitochondria precipitate was re-suspended with 50 μl Incubation buffer 2, combined with dNTP containing 1 μCi alpha-32p-dCTP, and incubated at 37° C. for 3 h;

2. Total cellular DNA was collected using a Total DNA extraction kit (Tiangen);

3. Digestion was performed with BstUI restriction enzyme overnight at 65° C.;

4 The digestion product was subjected to 5% acrylamide gel electrophoresis. The gel was removed afterwards, dried in the hood, detected with autoradiography exposure, the results of which shown in FIG. 2B. The “positive” lane represents the product obtained from the mitochondrial DNA from the mitochondrian isolated from the NIH3T3 cells after isotope incorporation. The “assembly” lane represents the product obtained from the mitochondrial DNA after electroporation after isotope incorporation. The “negative” lane represents the product obtained from only water after isotope incorporation.

### Example 4

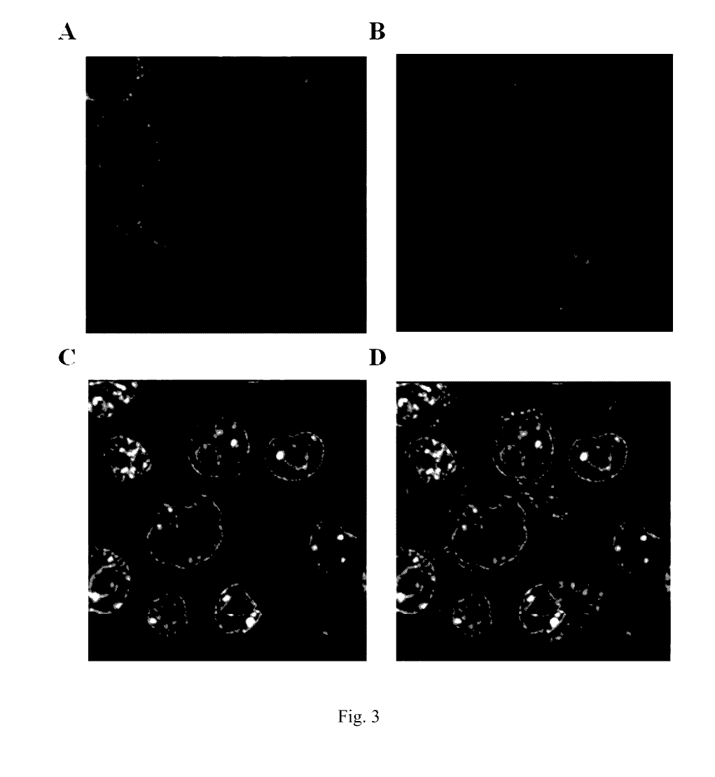

In this embodiment, the process of incorporating the synthetic mitochondria containing GFP-COX-I fusion gene into mouse macrophage cell line by endocytosis is described, with the results shown in FIG. 3.

(1) Mouse macrophage cell line RAW264.7 cell culture medium (high glucose DMEM (commercially available from Hyclone, Item No: SH30022.01B); 10% fetal bovine serum (commercially available from GIBCO, Item No: 16000-044); double antibody (including 10000 U/ml penicillin and 10000 μg/ml streptomycin, 1:100 dilution for use, available from GIBCO, Item No: 15140-122)). Cells were cultured in culture dishes according to conventional methods.

(2) The precipitated synthetic mitochondria obtained after electroporation as illustrated in Example 3 were re-suspended with 200 μL RAW264.7 cell culture medium. Aliquots of the suspension were added to culture dishes and incubated under 37° C. and 5% CO₂ in a water-saturated closed incubator. GFP-expressing mouse macrophage cells containing the exogenous mitochondria could be observed in 12 hours after endocytosis. The culture was further incubated for 12 hours and observations were made for detection, the results shown in FIG. 3.

The results clearly indicate that our design of GFP-COX I fusion gene is able to properly express in mitochondria. In FIG. 3, FIG. 3A shows the staining results with a mitochondria-specific dye, Mitotracker Red. FIG. 3B is the GFP detection results. FIG. 3C is the DAPI staining results, indicating the position of the nucleus. FIG. 3D is the three-color overlay.

In this embodiment, it is confirmed that the designed GFP-COX I fusion gene can be stably expressed in macrophages. Of course, mitochondria obtained through isolation that are not genetically altered (e.g., isolated or synthetic mitochondrion, the DNA sequences of which are wild-type mammalian mitochondrial DNA) can also be stably expressed in macrophages after entering macrophages by endocytosis.

The detailed embodiments described herein are only for the purpose of illustrating the present invention, and are not intended to limit the scope of the present invention in any way. It would be understand by a person skilled in the art that various changes and modifications can be made to the embodiments described herein without departing from the scope and spirit of the present invention. Such changes and modifications are contemplated by the present invention, the scope of which should only be defined by the following claims.

1. A mammalian cell comprising an exogenous mitochondrion, wherein the cell has the function of endocytosis.

2. The mammalian cell according to claim 1, wherein the exogenous mitochondrion is a synthetic mitochondrion obtained by introducing an exogenous DNA into a mitochondrion or an empty mitochondrial shell.

3. The mammalian cell according to claim 2, wherein the exogenous DNA is a synthetic mitochondrial DNA.

4. The mammalian cell according to claim 3, wherein the synthetic mitochondrial DNA is a mammalian cellular mitochondrial DNA with artificial gene modification.

5. The mammalian cell according to claim 1, wherein the exogenous mitochondrion is an isolated mammalian cellular mitochondrion.

6. The mammalian cell according to claim 1, wherein the cell is a macrophage.

7. Method for preparing a mammalian cell comprising an exogenous mitochondrion of claim 1, comprising:
adding the obtained exogenous mitochondrion to a cell culture system comprising a mammalian cell with endocytosis function and continuing culturing to obtain the cell comprising the exogenous mitochondrion.

8. The method according to claim 7, wherein
the mammalian cell with endocytosis function is a macrophage; and
the culturing conditions are 36-38° C. in temperature and 4.5-5.5% CO₂ in a water-saturated closed incubator.

9. A synthetic mitochondrion obtained by introducing an exogenous DNA into a mitochondrion or an empty mitochondrial shell.

10. Method for preparing the synthetic mitochondrion of claim 9, comprising:
obtaining an exogenous DNA;
preparing an empty mitochondrial shell, or obtaining a mitochondrion by isolation; and
introducing the exogenous DNA into the empty mitochondrial shell or into the isolated mitochondrion via eletroporation to obtain the synthetic mitochondrion.

11. The method according to claim 10, wherein
the exogenous DNA is an isolated wild-type DNA; or
the exogenous DNA is a synthetic mitochondrial DNA prepared by the following steps:
designing a new DNA sequence composition based on a mammalian mitochondrial DNA sequence using at least one gene modification means selected from the group consisting of gene introduction, gene knockout, site-directed mutagenesis, and gene rearrangement as needed, or, obtaining a wild-type DNA sequence composition; and
synthesizing DNA fragments of 50 bp to 60 bp according to the DNA sequence composition and connecting the synthesized DNA fragments with Gibson isothermal one-step method to obtain the synthetic mitochondrial DNA.

12. Method for introducing exogenous mitochondria into a mammalian cell, comprising:
obtaining an exogenous mitochondria; and
adding the exogenous mitochondrion to a cell culture system comprising a mammalian cell with endocytosis function and continuing culturing to obtain the cell comprising the exogenous mitochondrion.

13. The method according to claim 12, wherein the exogenous mitochondrion is an isolated mammalian cellular mitochondrion.

14. The method according to claim 12, wherein the exogenous mitochondrion is a synthetic mitochondrion obtained by introducing an exogenous DNA into a mitochondrion or an empty mitochondrial shell.

15. The method according to claim 14, wherein the exogenous DNA is an isolated wild-type DNA.

16. The method according to claim 14, wherein the exogenous DNA is a synthetic mitochondrial DNA, which is prepared by the following steps:
designing a new DNA sequence composition based on a mammalian mitochondrial DNA sequence using at least one gene modification means selected from the group consisting of gene introduction, gene knockout, site-directed mutagenesis, and gene rearrangement as needed, or, obtaining a wild-type DNA sequence composition; and
synthesizing DNA fragments of 50 bp to 60 bp according to the DNA sequence composition and connecting the synthesized DNA fragments with Gibson isothermal one-step method to obtain the synthetic mitochondrial DNA.

17. The method according to claim 12, wherein the mammalian cell is a macrophage.

18. The method according to claim 17, wherein the culturing conditions are 36-38° C. in temperature and 4.5-5.5% CO₂ in a water-saturated closed incubator.


SEQUENCE LISTING (1 sequence)

SEQ ID NO: 1 - DNA, 729 bp, Artificial Sequence
  Synthetic Mitochondria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggccgccgccacc